(19) United States
(12) Patent Application Publication
(10) Pub. No.: US 2022/0000591 A1
(43) Pub. Date: Jan. 6, 2022

(54) INTRAORAL MEASUREMENT DEVICE

(71) Applicant: Konica Minolta, Inc., Tokyo (JP)

(72) Inventors: Masayuki IIJIMA, Okazaki-shi (JP); Takahiro MATSUO, Toyokawa-shi (JP); Atsushi NAGAOKA, Okazaki-shi (JP)

(21) Appl. No.: 17/358,833

(22) Filed: Jun. 25, 2021

(30) Foreign Application Priority Data
Jul. 2, 2020 (JP) 2020-114530

(51) Int. Cl. A61C 9/00; A61B 1/24; A61B 5/00

(52) U.S. Cl. CPC A61C 9/0053; A61B 5/682; A61B 1/24

(57) ABSTRACT

Provided is an intraoral measurement device including a plurality of optical measurement systems, each of which measures a form of an intraoral object to be measured, and a hardware processor. The plurality of optical measurement systems respectively measure forms of intraoral areas different from each other. The hardware processor calculates the form of the intraoral object to be measured based on information obtained from the plurality of optical measurement systems.

### CROSS-REFERENCE TO RELATED APPLICATIONS

The entire disclosure of Japanese Patent Application No. 2020-114530 filed on Jul. 2, 2020 is incorporated herein by reference in its entirety.

### BACKGROUND

### Technological Field

The present disclosure relates to an intraoral measurement device.

### Description of the Related Art

In recent years, devices of three-dimensional intraoral measurement have come into use as an alternative to molding in dentistry. In obtaining three-dimensional image data of the whole oral cavity with such a device, multiple images obtained by moving the device inside the oral cavity are conjoined with one another to generate image data of the whole oral cavity. Multiple images are conjoined by matching singular points (ex. uneven portions) in each image.

However, errors may occur in conjoining two images depending on the precision and accuracy of the image data. Such errors are accumulated in image data of the whole oral cavity where multiple images are conjoined.

In a technique disclosed in JP 2019-170608 A as a countermeasure for this, an auxiliary instrument with multiple identifiable identification units on a sheet is installed in the oral cavity of a patient for reference of alignment.

### SUMMARY

However, in the technique disclosed in JP 2019-170608 A, it is necessary to install the auxiliary instrument inside the oral cavity of a patient, which increases burden on the patient. There may still be errors in conjoining depending on the precision of obtained image data.

The present invention has been conceived in view of the above circumstances and has an object of improving the accuracy of measurement of the intraoral form.

To achieve at least one of the abovementioned objects, an intraoral measurement device reflecting one aspect of the present invention includes:

a plurality of optical measurement systems, each of which measures a form of an intraoral object to be measured; and

a hardware processor;

wherein the plurality of optical measurement systems respectively measure forms of intraoral areas different from each other,

wherein the hardware processor calculates the form of the intraoral object to be measured based on information obtained from the plurality of optical measurement systems.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows a configuration of a main body of an intraoral measurement device in an embodiment;

FIG. 2 is a block diagram showing a schematic control configuration of the intraoral measurement device in the embodiment;

FIG. 3A is an explanatory diagram of actions of the intraoral measurement device in the embodiment;

FIG. 3B is an explanatory diagram of measurement of the entire oral cavity by a single optical measurement system;

FIG. 4 shows a configuration of a main device of the intraoral measurement device in Modification 1;

FIG. 5 is a block diagram showing a schematic control configuration of the intraoral measurement device in Modification 1;

FIG. 6A is an explanatory diagram of an extended state of a main body of the intraoral measurement device in Modification 2;

FIG. 6B is an explanatory diagram of a bent state of a main body of the intraoral measurement device in Modification 2;

FIG. 7A is an explanatory diagram of one state of a main body of the intraoral measurement device in Modification 2; and

FIG. 7B is an explanatory diagram of another state of a main body of the intraoral measurement device in Modification 2.

### DETAILED DESCRIPTION OF THE EMBODIMENTS

Hereinafter, an embodiment of the present invention is described with reference to the drawings. However, the scope of the present invention is not limited to the disclosed embodiment.

### [Configuration of Intraoral Measurement Device]

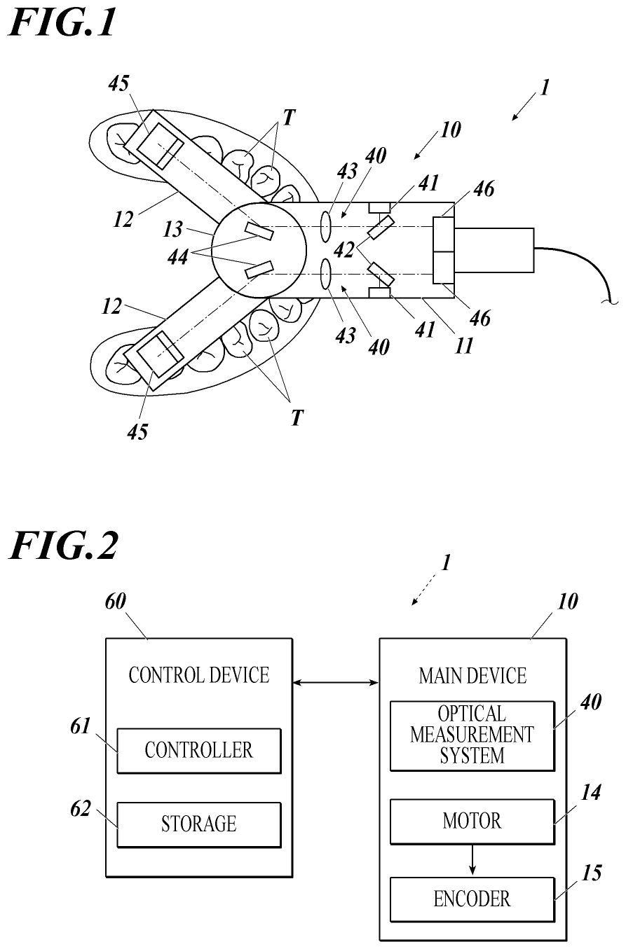

FIG. 1 shows a configuration of a main body 10 of an intraoral measurement device 1 in this embodiment.

The intraoral measurement device 1 mainly measures a three-dimensional form of an oral cavity/intraoral object to be measured of a human body. As shown in FIG. 1, the intraoral measurement device 1 includes a main device (body) 10.

The main body 10 is a part to be inserted into the oral cavity. The main body 10 houses, in its interior space, two optical measurement systems 40 individually for measuring the oral cavity three-dimensionally.

Each of the optical measurement systems 40 includes a laser light source 41, a half mirror 42, a condensing lens 43, a first mirror 44, a second mirror 45, and a light receiving sensor 46. In the optical measurement system 40, light emitted from the light source 41 is reflected on the half mirror 42, passes through the condensing lens 43, and is reflected on the first mirror 44 and the second mirror 45. Thereafter, the light is transmitted to the measurement object (ex. tooth T) in the oral cavity through a translucent window at the end of the main body 10 not shown in the drawings. At least part of the light is reflected on the measurement object and enters the main body 10 through the translucent window. Then, the light is received by the light receiving sensor 46 via the second mirror 45, the first mirror 44, the condensing lens 43, and then the half mirror 42. The form of the measured object inside the oral cavity is measured based on the optical information of the received light. As described later, the reflection on the first mirror 44 is omitted depending on the state of the main body 10.

The specific configurations of the optical measurement systems 40 are not limited as long as the optical cavity can be measured three-dimensionally.

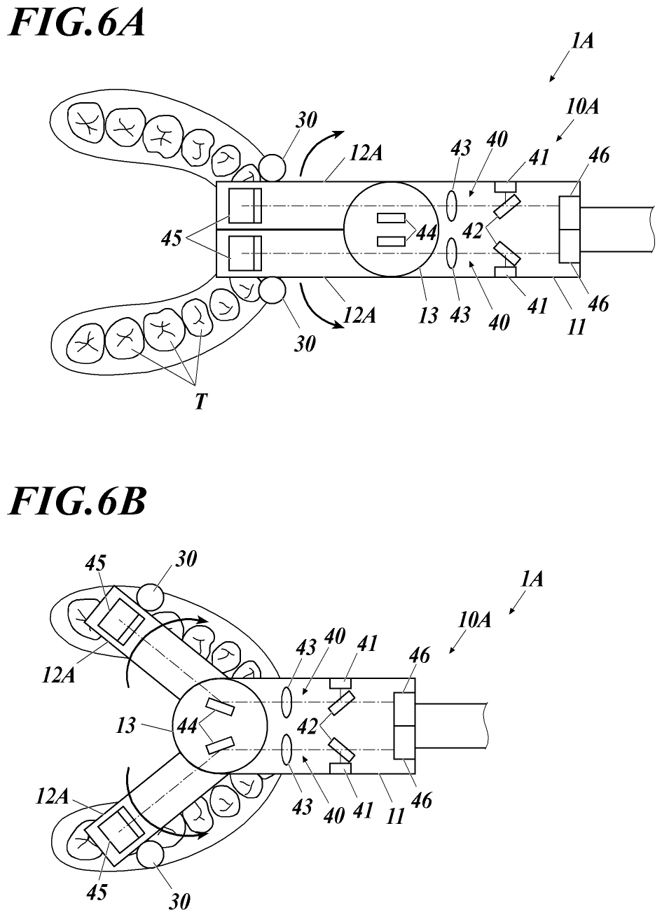

The main body 10 includes abase 11 and two arms 12. The base end of each of the two arms 12 is connected to the leading end of the base 11 via an elbow 13. The elbow 13 supports the two arms 12 rotatably so that the ends of the two arms 12 can approach and separate from each other on the same plane. The two arms 12 are rotatable in a predetermined angular range from the extended state where the ends of the two arms are close to each other along the longitudinal direction of the base 11 (see FIG. 6A) to the bent state where the ends of the two arms are separated (see FIG. 6B). The two arms 12 are rotated by the motor 14 incorporated in the elbow 13 (see FIG. 2) in the same amount. The two arms 12 may be individually rotated by separate motors.

In this description, the side inserted into the oral cavity ahead is referred to as the “leading end” side (left side of FIG. 1), and the side opposite to the leading end is referred to as the “base end” side.

The light source 41, the half mirror 42, the condensing lens 43, and the receiving light sensor 46, each component in a pair with another, of the two optical measurement systems 40 are arranged inside the base 11. The pair of the light receiving sensors 46 among those are arranged side by side at the base end of the base 11, and the pair of the half mirrors 42 and the pair of the condensing lenses 43 are arranged in the written order toward the leading end from the corresponding light receiving sensors 46. In this way, the pairs of the light receiving sensors 46, the half mirrors 42, and the condensing lenses 43 are arranged in series in the written order from the base end to the leading end, and the components in each pair are arranged side by side in the width direction of the base 11 (the up-down direction in FIG. 1). The two light sources 41 are each arranged on the side by the corresponding half mirror 42.

The second mirror 45 is arranged inside each of the arm 12. The second mirror 45 is arranged at the leading end of the arm 12 in a direction in which the light from the base end is reflected orthogonally to the rotation plane of the concerning arm 12 (the direction orthogonal to the sheet face in FIG. 1). When the arm 12 is extended, the second mirror 45 in its inside is positioned in a straight line connecting the corresponding receiving light sensor 46, the half mirror 42, and the condensing light lens 43.

The pair of the first mirrors 44 of the two optical measurement systems 40 are arranged inside the elbow 13. Each of the first mirrors 44 is arranged in a straight line connecting the corresponding receiving light sensor 46, the half mirror 42, and the condensing lens 43. Each of the first mirrors 44 rotates along with the rotation of the arm 12 that houses the corresponding mirror 45, and changes its direction by the rotation so as to reflect the light from the condensing lens 43 toward the second mirror 45. However, when the corresponding arm 12 is extended (or bent within a predetermined angle), the first mirror 44 is along the concerning arm 12, and the light from the condensing lens 43 enters the second mirror 45 without reflection on the first mirror 44 (see FIG. 6A).

FIG. 2 is a block diagram showing a schematic control configuration of the intraoral measurement device 1.

As shown in FIG. 2, the intraoral measurement device 1 includes a control device 60.

The control device 60 is connected to the main body 10 via a cable not shown in the drawings, and centrally controls the intraoral measurement device 1 according to the user's operation, for example. More specifically, the control device 60 includes a controller 61 (hardware processor) and a storage 62.

The storage 62 stores various programs for operations of the intraoral measurement device 1 and various kinds of data, such as information obtained by the optical measurement system 40.

The controller 61 controls the operation of the body 10 to measure the three-dimensional form in the oral cavity in accordance with the programs stored in the storage 62. Specifically, the controller 61 drives the motor 14 to rotate the two arms 12, obtains the drive amount of the motor 14 from the encoder 15 connected to the motor 14 (namely, the rotation amount of the pair of the arms 12), and controls the actions of the optical measurement system 40 so as to measure the three-dimensional form of the oral cavity.

### [Actions of Intraoral Measurement Device]

Next, the actions of the intraoral measurement device 1 are explained.

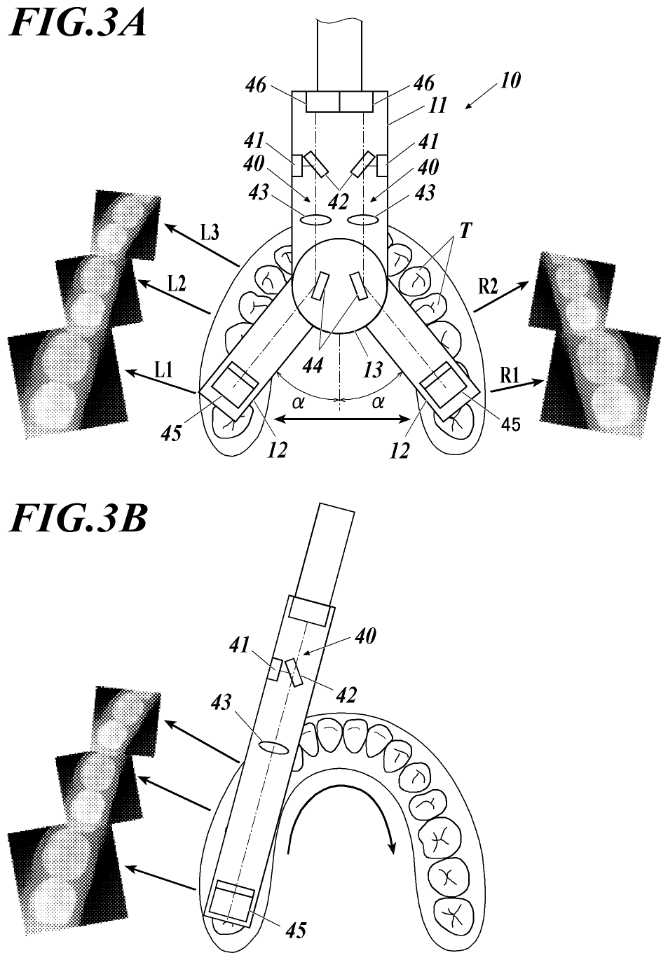

FIGS. 3A and B are an explanatory diagram of the actions of the intraoral measurement device 1 in measurement of the form of the oral cavity.

In measurement of the form of the oral cavity, the main body 10 is inserted into the oral cavity from the leading end side. At this time, as shown in FIG. 3A, the controller 61 rotates the two arms 12 by driving the motor 14 from the extended state and causes the leading ends of the arms 12 to face the back teeth. The controller 61 gradually closes the two arms 12 by driving the motor 14 while the main body 10 is manually moved from the back teeth side toward the front teeth side, and measures the row of teeth on the left and right sides individually and integrally by the two optical measurement systems 40. The direction of measurement in the oral cavity is not limited, and the row of teeth may be measured from the front teeth side toward the back teeth side while the two arms 12 are gradually opened.

The controller 61 causes the light receiving sensor 46 to receive the light emitted from the light source 41 and reflected on a tooth T in the oral cavity in each of the two optical measurement systems 40 and obtains the optical information on the light received by the light receiving sensor 46. The controller 61 generates three-dimensional image data based on the information obtained from the light receiving sensor 46.

In this way, multiple pieces of image data obtained by individual measurement of measured positions L1, L2, . . . on the left side and measured positions R1, R2, . . . on the right side by the two optical measurement systems 40. Here, the pieces of image data are generated so that the imaged ranges of the measured positions next to each other (partially) overlap.

Next, the controller 61 conjoins the generated pieces of image data with each other. Here, the controller 61 connects two pieces of image data of the measured positions next to each other so that the identical singular points match each other over and over, and conjoins the multiple pieces of image data.

The singular points on the images are not particularly limited as long as the tooth T can be identified from the image data, and an uneven portion or an external form may be used. In this way, one integral piece of image data of the entire row of the teeth in the oral cavity is generated.

As described above, in this embodiment, the form of the oral cavity is measured by the two optical measurement systems 40 individually. This makes it possible to decrease the number of image calculations by each of the optical measurement systems 40, which further suppresses accumulation of errors in conjoining, as shown in FIG. 3B, in comparison to measurement of the entire oral cavity by a single optical measurement system 40. This also makes it possible to shorten the time for calculation processing.

In conjoining multiple pieces of image data, the irradiation target position data may also be used for positioning adjustment in addition to the singular points on images. The irradiation target position data is information on the positional relations of the two irradiation target points at the tips of the two arms 12. The controller 61 calculates the irradiation target position data based on the distance between the rotation center and the irradiated positions of the arms 12 and the rotation amount of the two arms 12 obtained from the encoder 15 (the angle α from the extended state), and stores the data associated with the image data obtained at the above-mentioned rotation amount in the storage 62. The above-mentioned distance on the arms 12 is a measured value obtained in advance or a design value.

The controller 61 corrects the positioning in the image data based on the obtained irradiation target points when conjoining the image data of the row of teeth on the left and right respectively obtained by the two optical measurement systems 40. This makes it possible to generate more accurate image data of the entire oral cavity.

### Technical Effects of Embodiment

As described hereinbefore, the intraoral measurement device 1 in this embodiment measures forms of different areas of the oral cavity with the two optical measurement systems 40 and calculates the form of the oral cavity based on the information obtained by the two optical measurement systems 40.

This makes it possible to decrease the number of image calculations by each of the optical measurement systems 40 and suppress accumulation of errors in conjoining, in comparison to conventional measurement by a single optical measurement system 40.

The intraoral measurement device 1 in this embodiment conjoins multiple pieces of image data based on singular points on the image data and irradiation target position data on positional relations between two irradiation target positions.

This makes it possible to generate more accurate image data of the entire oral cavity.

In the intraoral measurement device 1 in this embodiment, the irradiation target position data is calculated based on the rotation angle α of the two arms 12 obtained from the encoder 15.

This makes it possible to suitably calculate the irradiation target position data and further suitably generate more precise image data of the entire oral cavity.

### Modification 1

Next, an intraoral measurement device 2 in Modification 1 of this embodiment is explained.

The intraoral measurement device 2 in this modification is different from the above-described intraoral measurement device 1 in that the two arms of the main body do not rotate but move translationally. Hereinafter, the difference(s) is mainly described. The components similar to the above-described embodiment are given the same reference numerals and description thereof is omitted.

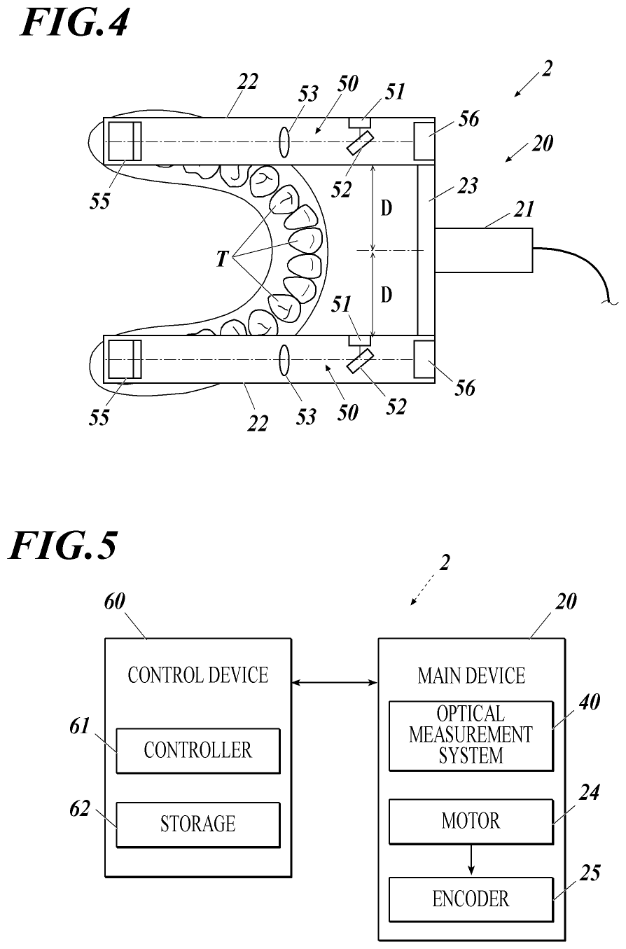

FIG. 4 shows a configuration of the main device 20 of the intraoral measurement device 2.

As shown in FIG. 4, the intraoral measurement device 2 includes the main body 2.

The main body 20 includes two optical measurement systems 50 inside for individually measuring the oral cavity three-dimensionally.

Each of the optical measurement systems 50 includes a light source 51, a half mirror 52, a condensing lens 53, a mirror 55, and a light receiving sensor 56. These components function similarly to the light source 41, the half mirror 42, the condensing lens 43, the second mirror 45, and the light receiving sensor 46, respectively.

The main body 20 includes a base 21 and two arms 22.

The base 21 includes a rail 23 along the width direction of the main body 20 (the up-down direction in FIG. 4).

The two arms 22 are arranged in series in the width direction, extending in the direction orthogonal to the width direction. The base ends of the two arms 22 are connected to the rail 23. The rail 23 supports the two arms 22 movably in the width direction so that the two arms 22 can approach and separate from each other. The two arms 22 are moved in the same amount by a motor 24 (see FIG. 5) incorporated in the base 21. The two arms 22 may be individually rotated by separate motors.

The two optical measurement systems 50 are individually housed in the two arms 22. Specifically, the light receiving sensor 56, the half mirror 52, the condensing lens 53, and the mirror 55 are arranged in the written order in series from the base end to the leading end inside each of the arms 22, and the receiving light sensor 56 is arranged at the base end part and the mirror 55 at the leading end part. The light source 51 is arranged on the side by the half mirror 52.

FIG. 5 is a block diagram showing a schematic control configuration of the intraoral measurement device 2.

As shown in FIG. 5, the intraoral measurement device 2 includes a control device 60.

The control device 60 is connected to the main body 20 via a cable not shown in the drawings, and centrally controls the intraoral measurement device 1 according to the user's operation, for example. More specifically, the control device 60 includes a controller 61 (hardware processor) and a storage 62.

The control device 60 functions similarly to the corresponding device in the above-described embodiment.

The controller 61 in this modification moves the two arms 22 by driving the motor 24 instead of the motor 14 in the above-described embodiment, and obtains the drive amount of the motor 24 from the encoder 25 connected to the motor 24 (namely, the movement amount of the two arms 22).

The effects similar to those of the above-described embodiment can be achieved by the intraoral measurement device 2 configured as described above.

The irradiation target position data for positioning adjustment of the image data may be calculated based on the movement amount of the two arms 22 obtained from the encoder 25 (the movement distance D from the center position: see FIG. 4) instead of the rotation angle α of the two arms 12 in the above-described embodiment.

### Modification 2

Next, the intraoral measurement device 1A in Modification 2 of this embodiment is explained.

FIGS. 6A and 6B show a configuration of a main body 10A of the intraoral measurement device 1A.

As shown in FIGS. 6A and 6B, the main body 10A of the intraoral measurement device 1A includes two arms 12A instead of the two arms 12.

Each of the two arms 12A includes a guide member 30 at the leading end on the external side in the rotation direction. Each of the arms 12A are configured similarly to the arms 12 in the above-described embodiment in other respects.

The guide member 30 is composed of a flexible elastic body having sufficient safety for the human body, for example. As the guide member 30 is in contact with a surrounding part including the measured object in the mouth (ex. gum, lip, etc.), it is possible to stabilize the movement of the main body 10A including rotation of the two arms 12A and improve the positioning accuracy of the image data. Here, the arms 12A may be moved by the guide member 30 in contact with a part in the mouth instead of being driven by the motor 14.

The position and form of the guide member 30 are not particularly limited as long as the guide member 30 is in contact with a surrounding part including the measured object to stabilize the main body 10A.

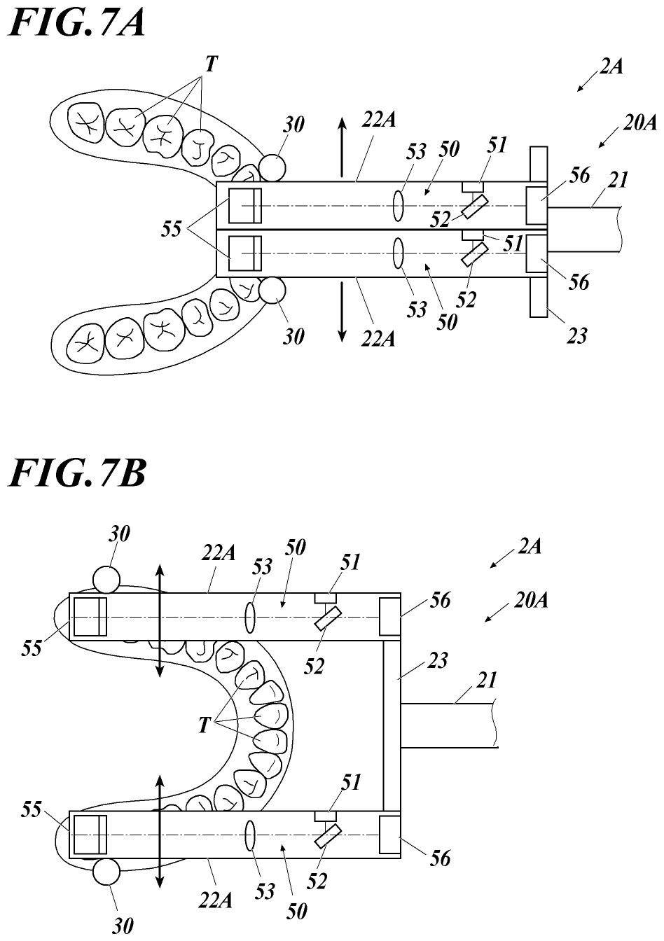

As shown in FIGS. 7A and 7B, the guide member 30 may be included in the intraoral measurement device 2 in the above-described Modification 1.

On the main body 20A of the intraoral measurement device 2A including the guide member 30, the guide member 30 is arranged on the external side of each of the two arms 22A. The similar effects as described above can be archived thereby.

### MISC

Hereinbefore, an embodiment of the present invention has been described. However, the present invention is not limited to the above embodiment and can be appropriately modified without departing from the scope of the present invention.

For example, the above-described embodiment and modifications, the two arms as movable parts are driven by a motor, but the two arms may be manually moved.

The driving means (driving mechanism) for the movable parts or the means of detecting the movement amount of the movable parts are not limited to a motor or an encoder.

The two arms that rotate or move translationally are described as an exemplary movable part according to the present invention, but the movable state is not limited as long as the movable part moves the irradiation target positions.

Further, the movable part is not necessarily provided (two arms are not necessarily movable) as long as two optical measurement systems are provided. In that case, the irradiation target position data on the positional relations of the two irradiation target positions is measured or calculated beforehand and stored in the storage.

Three or more optical measurement systems may be used. For example, four optical measurement systems consisting of two systems that measures the row of maxillary teeth and two systems that measures the row of mandibular teeth may be used. In that case, the two optical measurement systems for the mandibular teeth, and those inverted upside down for the maxillary teeth are arranged in parallel in the up and down direction of the main body.

Such multiple optical measurement systems may share the components as long as each can measure the form individually. However, each system need to include at least a light source by itself.

What is claimed is:

1. An intraoral measurement device comprising:
a plurality of optical measurement systems, each of which measures a form of an intraoral object to be measured; and
a hardware processor;
wherein the plurality of optical measurement systems respectively measure forms of intraoral areas different from each other,
wherein the hardware processor calculates the form of the intraoral object to be measured based on information obtained from the plurality of optical measurement systems.

2. The intraoral measurement system according to claim 1,
wherein each of the plurality of optical measurement systems comprises:
a light source;
an optical element that condenses light emitted from the light source and leads the light to the object to be measured; and
a light receiving sensor that receives the light reflected on the intraoral object to be measured.

3. The intraoral measurement system according to claim 2,
wherein the hardware processor:
generates multiple pieces of three-dimensional image data based on information obtained from the light receiving sensor;
conjoins the pieces of image data based on singular points of the respective pieces of the image data and irradiation target position information on a positional relation of positions irradiated by the plurality of optical measurement systems.

4. The intraoral measurement device according to claim 3, further comprising:
a storage that stores the irradiation target position information in advance.

5. The intraoral measurement device according to claim 3, further comprising:
a movable pan that moves the positions irradiated by the plurality of optical measurement systems; and
a detector that detects a movement amount of the movable part,
wherein the hardware processor calculates the irradiation target position information based on the movement amount obtained from the detector.

6. The intraoral measurement device according to claim 5,
wherein the movable part comprises a guide member that is in touch with a mouth.
